(19) United States
(12) Patent Application Publication
(10) Pub. No.: US 2026/0289300 A1
(43) Pub. Date: Sep. 24, 2026

(54) TRAINING DEVICE, TRAINING METHOD, AND RECORDING MEDIUM

(71) Applicant: NEC Corporation, Tokyo (JP)

(72) Inventor: Chenhui HUANG, Tokyo (JP)

(73) Assignee: NEC Corporation, Tokyo (JP)

(21) Appl. No.: 19/564,342

(22) Filed: Mar. 12, 2026

(30) Foreign Application Priority Data
Mar. 24, 2025 (JP) 2025-048089

(51) Int. Cl. G06N 3/08; G06N 20/20; G16H 50/30

(52) U.S. Cl. CPC G06N 3/08; G06N 20/20; G16H 50/30

(57) ABSTRACT

In the training device, the training unit trains models having a common architecture by using different loss functions to generate a plurality of sub-models. The integration unit integrates the plurality of sub-models to generate a final model. By using the model trained by the training device to estimate disease risk, it is possible to support decision-making regarding the subject's lifestyle habits.

### INCORPORATION BY REFERENCE

This application is based upon and claims the benefit of priority from Japanese Patent Application 2025-048089, filed on Mar. 24, 2025, the disclosure of which is incorporated herein in its entirety by reference.

### TECHNICAL FIELD

The present disclosure relates to training of a machine learning model.

### BACKGROUND ART

Ensemble learning has been known as a method for improving prediction accuracy by combining a plurality of machine learning models. Patent document JP2022-131558A describes an ensemble learning system using a plurality of machine learning models.

### SUMMARY

The ensemble learning attempts to take advantage of various models and achieve more accurate prediction. However, the conventional ensemble learning uses models trained by training methods of different systems in combination, and does not particularly focus on a loss function.

One object of the present disclosure is to provide a training device capable of creating a highly accurate model by integrating models trained using different loss functions.

According to an example aspect of the present invention, there is provided a training device comprising:
a training means configured to train models having a common architecture by using different loss functions to generate a plurality of sub-models; and
an integration means configured to integrate the plurality of sub-models to generate a final model.

According to another example aspect of the present invention, there is provided a training method executed by a computer, comprising:
training models having a common architecture by using different loss functions to generate a plurality of sub-models; and
integrating the plurality of sub-models to generate a final model.

According to still another example aspect of the present invention, there is provided a program for causing a computer to execute processing including:
training models having a common architecture by using different loss functions to generate a plurality of sub-models; and
integrating the plurality of sub-models to generate a final model.

According to the present disclosure, it is possible to provide the training device capable of creating the highly accurate model by integrating the models trained using the different loss functions.

### BRIEF DESCRIPTION OF THE DRAWINGS

FIG. 1 schematically illustrates a basic concept of the present disclosure;

FIG. 2 illustrates the entire configuration of a training device according to the present disclosure;

FIG. 3 is a block diagram illustrating a hardware configuration of the training device;

FIG. 4 is a block diagram illustrating a functional configuration of the training device;

FIG. 5 illustrates exemplary loss functions;

FIG. 6 illustrates an exemplary configuration of a training unit;

FIG. 7 is a flowchart of training processing;

FIG. 8 illustrates another exemplary loss function;

FIG. 9 illustrates an exemplary probability distribution of training data;

FIG. 10 is an explanatory graph for hyperparameters in the another loss function;

FIG. 11 illustrates another exemplary configuration of a training unit;

FIG. 12 is a flowchart of another training processing;

FIG. 13 is a block diagram illustrating a configuration of another training device according to the present disclosure; and

FIG. 14 is a flowchart of processing in the another training processing.

### EXAMPLE EMBODIMENTS

Preferred example embodiments of the present disclosure will be described below with reference to the drawings. In the following description, for convenience of notation, a symbol given above a certain variable is expressed by a character next to the variable. For example, a variable “y” above which a symbol “∧ (hat)” is added is expressed as “y\hat{ }”.

### Basic Concept

The ensemble learning attempts to take advantage of various models and achieve more accurate prediction. However, the conventional ensemble learning uses models trained by training methods of different systems in combination, and does not particularly focus on a loss function.

Various loss functions used for training of deep learning models have various characteristics and focus on differences in a distribution of training data. Therefore, it is considered that a highly accurate model, suitable for various portions in the distribution of training data, can be generated by training models having the same architecture with different loss functions. Therefore, in the present disclosure, deep learning models of the same architecture are trained using different loss functions, and a plurality of obtained models are integrated to generate a highly accurate final model.

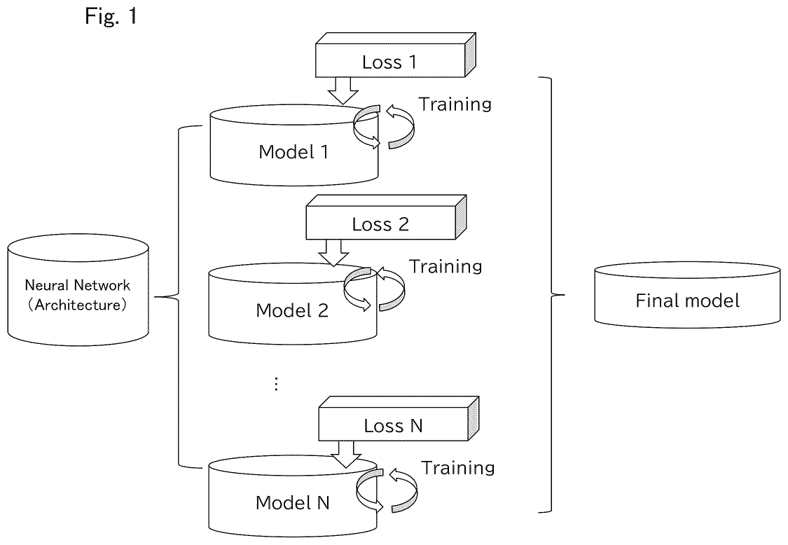

FIG. 1 schematically illustrates a basic concept of the present disclosure. A neural network of one architecture is prepared, and a plurality of models 1 to N is trained based on the neural network. That is, as a premise, the models are assumed to have the same architecture. Here, the “architecture” refers to a structure of the neural network, and specifically refers to the number of neurons, the number of layers, an array of parameters in the layers, a combination of the layers (order of the layers), hyperparameters, and the like constituting the neural network. Note that the parameter is also referred to as a coefficient, a weight, or the like. In the models 1 to N, the above elements are the same. Each of the models 1 to N corresponds to a sub-model.

The models 1 to N are trained using different loss functions, respectively. Thus, the plurality of models 1 to N that perform prediction focusing on different portions in a distribution of input data are obtained. Then, a final model is obtained by integrating the plurality of models 1 to N. Thus, the final model becomes a model adapted to a wider region in the distribution of input data, and the prediction adapted to characteristics of the input data becomes possible.

### First Example Embodiment

### Overall Configuration

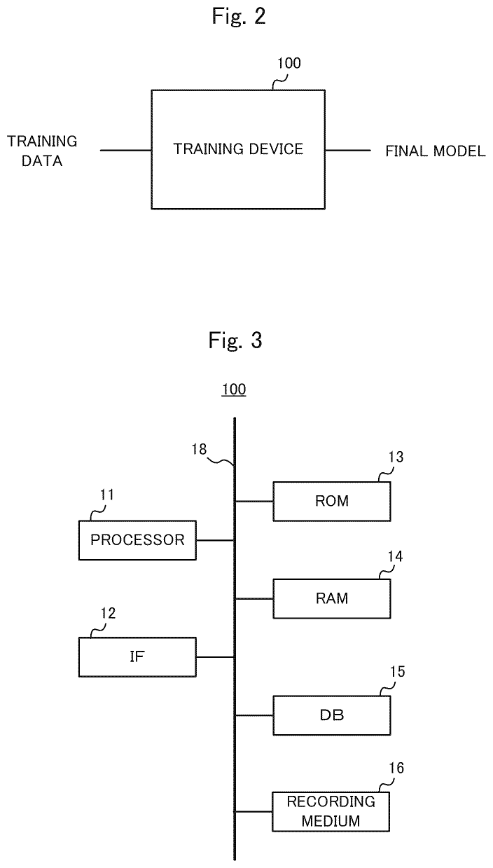

FIG. 2 illustrates an overall configuration of a training device according to a first example embodiment. A training device 100 trains the plurality of models described above based on training data and outputs the final model obtained by integrating the plurality of models. In the present example embodiment, it is assumed that the plurality of models and the final model are risk estimation models for estimating a disease risk of a subject based on data regarding health of the subject. Specifically, a data set of the training data used by the training device 100 includes input data and a true value (ground truth). The input data includes, for example, various pieces of data obtained by medical checkup or the like, such as height, weight, sex, blood pressure, body mass index (BMI), body fat percentage, neutral fat value, smoking status and amount, drinking status and amount, and the like of the subject. Note that the input data may be multimodal data, namely, a plurality of pieces of data in the above-described various types of data.

The risk estimation model generated as the final model by the training device 100 can be favorably applied to the medical or healthcare field. For example, the final model can be used to estimate the risk of any lifestyle-related disease, based on data obtained in a regular medical examination.

### Hardware Configuration

FIG. 3 is a block diagram illustrating a hardware configuration of the training device 100. As illustrated, the training device 100 includes a processor 11, an interface (IF) 12, a read only memory (ROM) 13, a random access memory (RAM) 14, a database (DB) 15, and a storage medium 16. The components are connected to each other via a bus 18, for example.

The processor 11 corresponds to a computer, such as a central processing unit (CPU), and executes a program prepared in advance to control the entire training device 100. Specifically, as the processor 11, a CPU, a graphics processing unit (GPU), a digital signal processor (DSP), a micro processing unit (MPU), a floating point number processing unit (FPU), a physics processing unit (PPU), a tensor processing unit (TPU), a quantum processor, a microcontroller, or a combination of these can be used.

In addition, the processor 11 loads a program stored in the ROM 13 or the storage medium 16 into the RAM 14 and executes each process coded in the program. The processor 11 functions as part of the training device 100 or the entirety of the training device 100. Specifically, the processor 11 performs training processing, which will be described later.

The IF 12 transmits and receives data to and from an external device. Specifically, the training device 100 receives, as training data, input data regarding the health of a plurality of persons through the IF 12. In addition, the training device 100 outputs a trained risk estimation model to a storage device or an external device through the IF 12.

The ROM 13 stores various programs to be executed by the processor 11. The RAM 14 is used as a working memory during execution of various types of processing by the processor 11.

The DB 15 stores various types of algorithms, data, and deep learning models for use when the training device 100 performs the training processing, which will be described later. In particular, the DB 15 stores a network architecture serving as the basis of a plurality of models, information on a plurality of loss functions, and the like.

The storage medium 16 is a non-volatile non-transitory storage medium such as a disk-shaped recording medium or a semiconductor memory. The storage medium 16 may be detachably attached to the training device 100. The storage medium 16 records various programs to be executed by the processor 11.

Note that, in addition to the above, the training device 100 may include a display device, such as a liquid crystal display, and an input device, such as a keyboard or a mouse. Such a display device and an input device are used, for example, by an operator for the training device 100.

### Functional Configuration

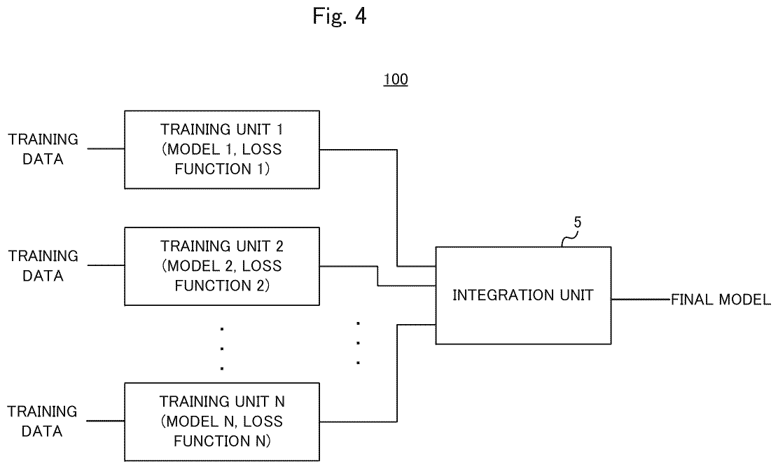

FIG. 4 is a block diagram illustrating a functional configuration of the training device 100. The training device 100 includes a plurality of training units 1 to N and an integration unit 5.

The training units 1 to N are examples of training means, and train a model of the same network architecture (hereinafter, also referred to as a “common architecture”) using different loss functions as described above. In the example of FIG. 4, the training unit 1 trains the model of the common architecture using Loss function 1 and generates Model 1. Similarly, the training unit 2 generates Model 2 of the common architecture using Loss function 2, and the training unit N generates Model N of the common architecture using Loss function N.

Each of the training units 1 to N outputs parameter groups of the generated models to the integration unit 5. The parameter group is a set of parameters set to the neural network constituting each model. As described above, since Models 1 to N are models of the common architecture, the parameter groups of the models have the same structure. Note that the parameters are coefficients and weights set for each layer of the neural network.

The integration unit 5 is an example of an integration means, and integrates the parameter groups output from the training units 1 to N to generate the final model. As described above, since Models 1 to N have the common architecture and structures of the parameter groups are the same, the integration unit 5 can generate a parameter group of the final model by calculating the parameter groups of the respective models. For example, the integration unit 5 can determine the parameter group of the final model by calculation such as averaging, simple addition, weighted addition, majority decision, or the like of the parameter groups of Models 1 to N. Then, the integration unit 5 outputs the final model including the obtained parameter group. Note that the integration unit 5 may output the parameter group of the final model instead of the final model.

### Loss Function

Next, examples of the loss function used by each training unit will be described.
(1) Examples of General Loss Functions

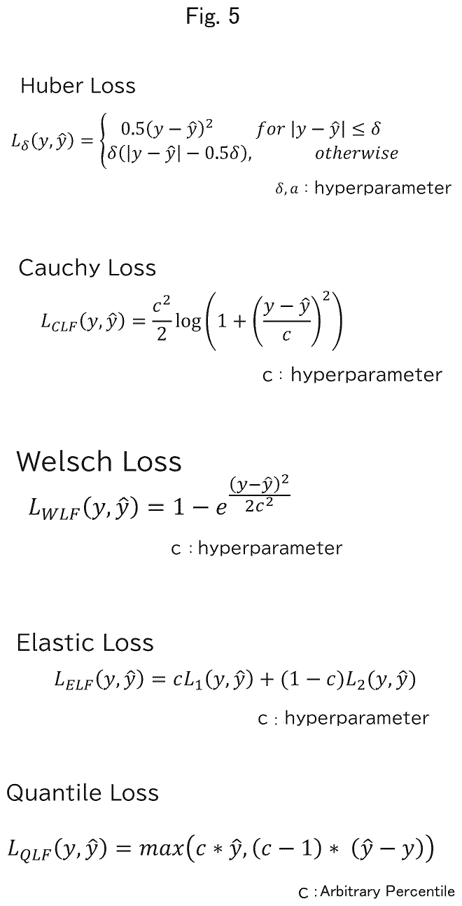

FIG. 5 illustrates exemplary general loss functions.

The Huber loss has the advantage of mean squared error (MSE) and the advantage of mean absolute error (MAE) in combination. The Huber loss switches a mean squared error and a mean absolute error depending on whether an absolute value |(y−y\hat{ })| of a difference between a true value y and a predicted value y\hat{ } is larger than a threshold δ.

The Cauchy loss is a robust loss function against outliers and thus is less affected by outliers as a characteristic. This loss function uses a difference between a true value and a predicted value.

The Welsch loss is also a robust loss function against outliers and suppresses the influence of outliers as a characteristic. This loss function also uses a difference between a true value and a predicted value.

The Elastic loss is also a robust loss function against outliers. This loss function also uses a difference between a true value and a predicted value.

The Quantile loss is used to minimize a prediction error to a particular quantile. This loss function divides a distribution by arbitrary percentile.

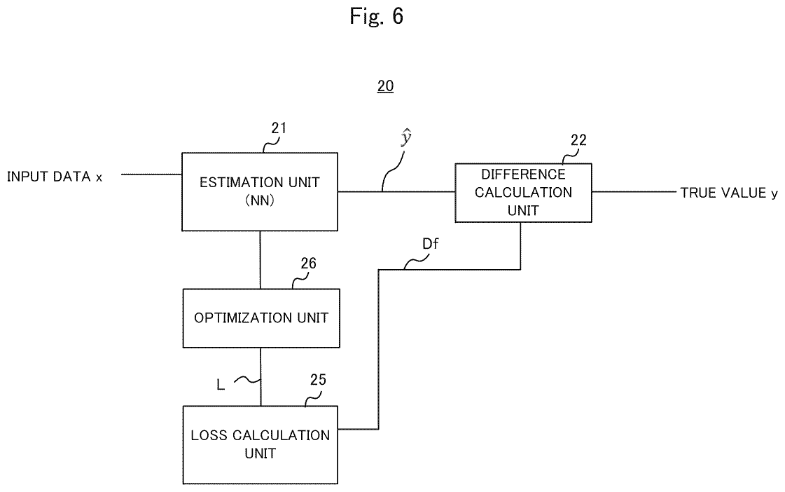

FIG. 6 illustrates a configuration of the training unit in a case where the above loss function is used. The training unit 20 includes an estimation unit 21, a difference calculation unit 22, a loss calculation unit 25, and an optimization unit 26. The estimation unit 21 includes a neural network (NN) that functions as a risk estimation model. Input data x included in a data set of training data is input to the estimation unit 21. The input data x corresponds to an explanatory variable. The estimation unit 21 estimates a risk from the input data x using the risk estimation model and outputs an estimated value y\hat{ } to the difference calculation unit 22. The difference calculation unit 22 calculates a difference Df between the estimated value y\hat{ } and a true value y and outputs the difference Df to the loss calculation unit 25. Note that the true value y corresponds to a response variable (objective variable) to the explanatory variable x.

The difference Df input from the difference calculation unit 22 is input to the loss calculation unit 25. The loss calculation unit 25 calculates a loss L using the loss function and outputs the loss L to the optimization unit 26. As the loss function, for example, any one of the loss functions illustrated in FIG. 5 can be used, but the loss function is not limited thereto. The optimization unit 26 optimizes parameters of the neural network included in the estimation unit 21 in such a way that the loss L input from the loss calculation unit 25 is minimized.

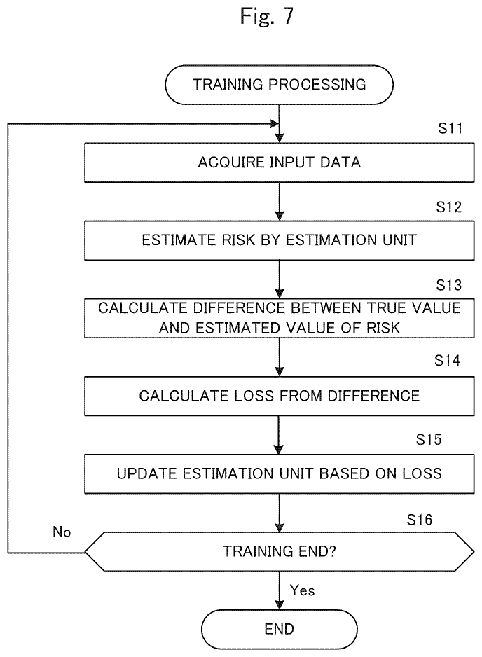

FIG. 7 is a flowchart of training processing in a case where a general loss function is used. This processing is achieved by the processor 11 illustrated in FIG. 3 executing a program prepared in advance and operating as the components illustrated in FIG. 6.

First, the estimation unit 21 acquires input data included in training data (step S11). Next, the estimation unit 21 estimates a risk from the input data using the risk estimation model including the neural network (step S12). Next, the difference calculation unit 22 calculates the difference between the true value and the estimated value of the risk (step S13). Next, the loss calculation unit 25 calculates a loss from a difference using any of the loss functions (step S14). Next, the optimization unit 26 updates the parameters of the neural network of the estimation unit 21 in such a way that the loss is reduced (step S15).

Next, the training unit 20 determines whether a predetermined training end condition is satisfied (step S16). Examples of the training end condition include a condition that a predetermined number of pieces of data prepared as the training data have been used, a condition that the loss L is equal to or less than a predetermined value, and a condition that the loss L has converged. In a case where the training end condition is not satisfied (step S16: No), the processing returns to step S11. On the other hand, in a case where the training end condition is satisfied (step S16: Yes), the training processing ends.

The training units 1 to N illustrated in FIG. 4 can generate Models 1 to N of the common architecture using different loss functions by the above training method.

(2) Loss Function Considering Probability Distribution of Training Data
The training unit may use a loss function considering a probability distribution of training data in addition to the general loss functions described above. Hereinafter, this loss function is also referred to as a “distribution-considered loss” or a “distribution-considered loss function”. The general loss functions aim to reduce the sum total of the differences between true values and estimated values in the entire distribution of training data. Thus, like medical examination data of unhealthy persons, a small number of pieces of data deviating greatly from the center of a distribution have a low contribution ratio to the sum total of losses in the entire distribution and thus tend to be neglected or ignored in training of an estimation model. On the other hand, the distribution-considered loss described hereinafter is a loss function adaptive to the distribution of training data by weighting a loss using a distribution characteristic of the training data.

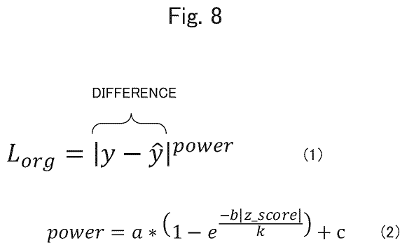

FIG. 8 illustrates an expression of the distribution-considered loss. A distribution-considered loss function L_{org} in Expression (1) uses the Minkowski distance and includes a difference and an exponent that exponentiates the difference. The exponent of the Minkowski distance is called a “power”. This power corresponds to a weight in the distribution-considered loss. The power indicates a deviation (degree of deviation) from the center of a probability distribution of training data, and is defined by a mean μ and a standard deviation σ as parameters of a probability density function indicating a normal distribution. The power can be expressed by Expression (2) using a distance z_score from the center of the probability distribution of training data. The distance z_score is calculated from the mean μ and the standard deviation σ. Note that a, b, and c are hyperparameters.

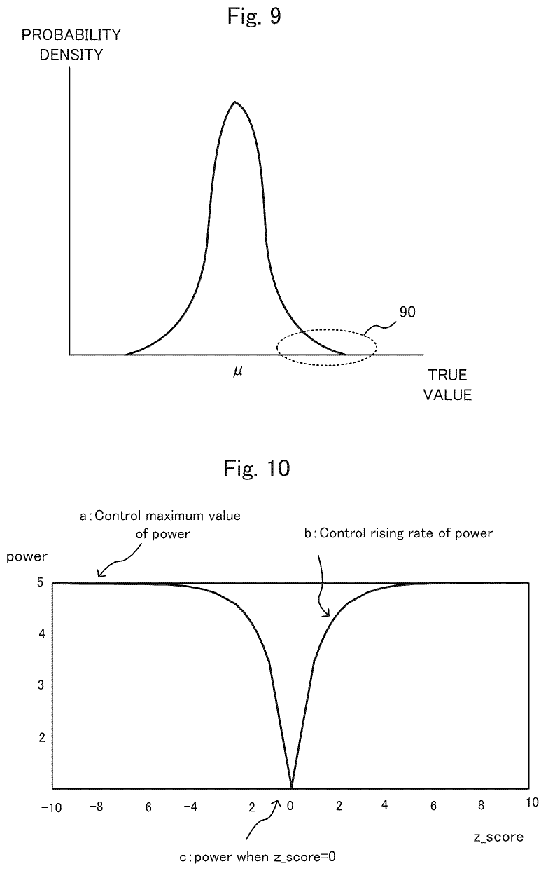

FIG. 9 illustrates an exemplary probability distribution of training data. Data of unhealthy persons in medical examination data gathers in a region 90 deviating from the center of the distribution (hereinafter, also referred to as an “outlier region”). In the outlier region 90, the distance from the center of the distribution is large and thus the value of the distance z_score is large. Thus, regarding the data in the outlier region 90, the value of the power expressed by Expression (2), namely, the weight is large and thus the loss L_{org} is large. Thus, the influence of the data in the outlier region on the loss L_{org} can be increased. in Expression (2), the probability distribution of training data is not necessarily accurately a normal distribution, and thus a skewness k may be taken into account, depending on a probability distribution of training data to be used.

FIG. 10 is an explanatory graph for the hyperparameters a, b, and c in Expression (2). In FIG. 10, the horizontal axis represents the distance z_score from the center of the probability distribution and the vertical axis represents the power. The hyperparameter a controls the maximum value of the power. The hyperparameter b controls the rising rate of the power. The hyperparameter c indicates the power in a case where the distance z_score is 0, namely, a starting point for the power. The hyperparameters a, b, and c are basically set by a user.

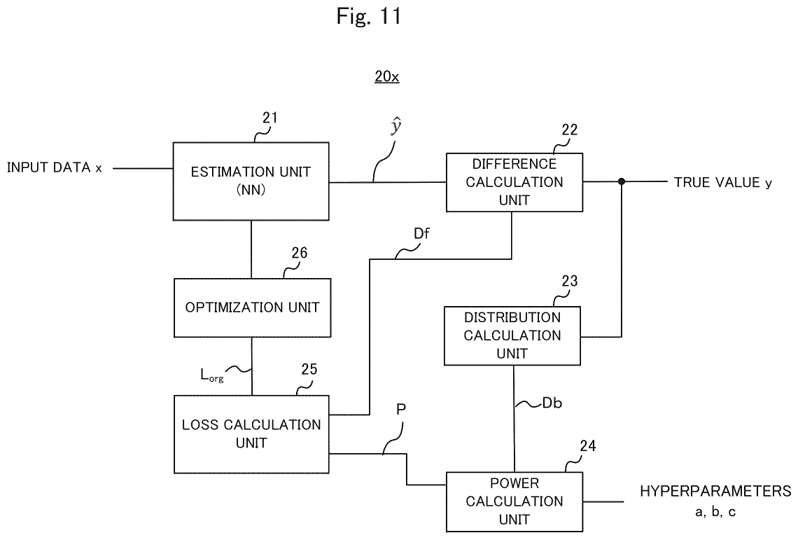

FIG. 11 illustrates a configuration of a training unit in a case where the distribution-considered loss is used. A training unit 20x includes the estimation unit 21, the difference calculation unit 22, a distribution calculation unit 23, a power calculation unit 24, the loss calculation unit 25, and the optimization unit 26. Note that the estimation unit 21 and the difference calculation unit 22 are similar to those in FIG. 6, and thus description thereof is omitted.

The distribution calculation unit 23 calculates a probability density function of the true value y of the training data to obtain a probability distribution. The distribution calculation unit 23 outputs, to the power calculation unit 24 and the loss calculation unit 25, a parameter Db that defines the obtained probability distribution. As an example, the parameter Db includes the mean μ and the standard deviation σ.

The hyperparameters a, b, and c are set to the power calculation unit 24. The power calculation unit 24 obtains the distance z_score from the parameter Db, calculates a power P based on Expression (2), and outputs the power P to the loss calculation unit 25.

Based on the difference Df input from the difference calculation unit 22 and the power P input from the power calculation unit 24, the loss calculation unit 25 calculates the loss L_{org} using the loss function of Expression (1) and outputs the loss L_{org} to the optimization unit 26.

The optimization unit 26 optimizes the parameters of the neural network included in the estimation unit 21 in such a way that the loss L_{org} input from the loss calculation unit 25 is minimized.

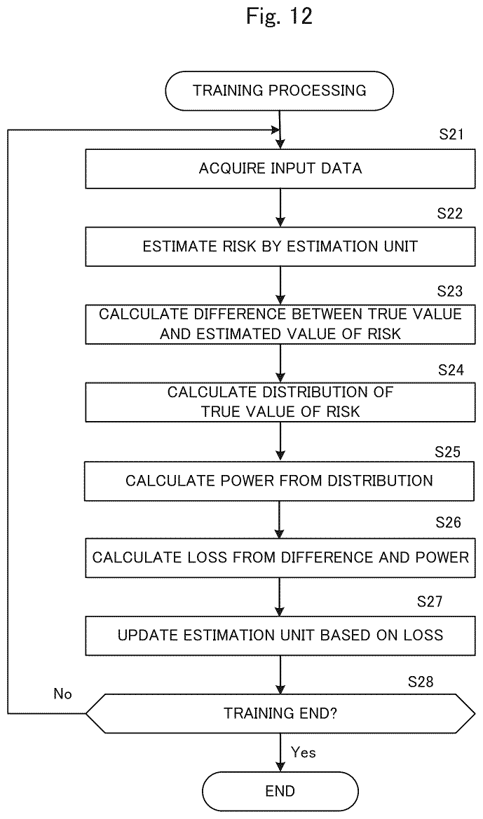

FIG. 12 is a flowchart of training processing in a case where the distribution-considered loss is used. This processing is achieved by the processor 11 illustrated in FIG. 3 executing a program prepared in advance and operating as the components illustrated in FIG. 11.

First, the estimation unit 21 acquires input data included in training data (step S21). Next, the estimation unit 21 estimates a risk from the input data using the risk estimation model including the neural network (step S22). Next, the difference calculation unit 22 calculates a difference between a true value and an estimated value of the risk (step S23). Next, the distribution calculation unit 23 calculates a probability distribution of the true value of the risk (step S24). Next, the power calculation unit 24 calculates a power using the probability distribution of the true value (step S25).

Next, the loss calculation unit 25 calculates a loss from the difference and the power (step S26). Next, the optimization unit 26 updates the parameters of the neural network of the estimation unit 21 in such a way that the loss is reduced (step S27).

Next, the training unit 20x determines whether a predetermined training end condition is satisfied (step S28). In a case where the training end condition is not satisfied (step S28: No), the processing returns to step S21. On the other hand, in a case where the training end condition is satisfied (step S28: Yes), the training processing ends.

### Effects

As described above, the training device of the first example embodiment trains a plurality of models of a common architecture using different loss functions to generate the plurality of models having different characteristics. Then, parameters of the plurality of generated models are integrated to generate a final model. Thus, the final model adaptive to various characteristics of input data can be constructed.

### Second Example Embodiment

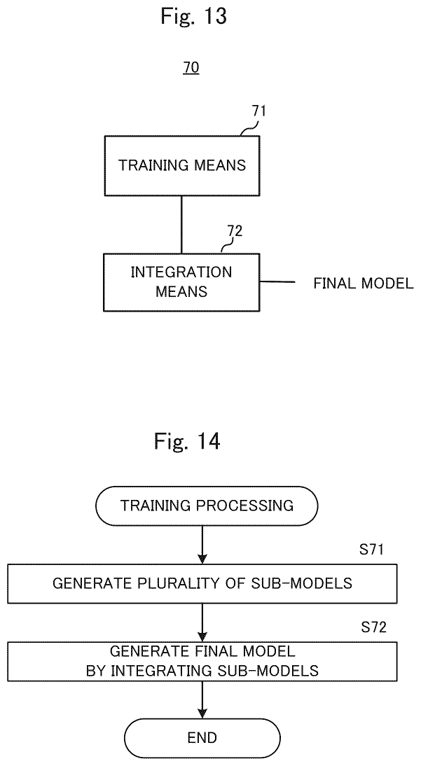

FIG. 13 is a block diagram illustrating a functional configuration of a training device according to a second example embodiment. A training device 70 includes a training means 71 and an integration means 72.

FIG. 14 is a flowchart of processing by the training device according to the second example embodiment. The training means 71 trains models having a common architecture by using different loss functions to generate a plurality of sub-models (step S71). The integration means 72 integrates the plurality of sub-models to generate a final model (step S72).

According to the training device 70 of the second example embodiment, it is possible to create a highly accurate model by integrating models trained using different loss functions.

Some or all of the example embodiments described above may also be described as, but are not limited to, the following supplementary notes.

### Supplementary Note 1

A training device comprising:
a training means configured to train models having a common architecture by using different loss functions to generate a plurality of sub-models; and
an integration means configured to integrate the plurality of sub-models to generate a final model.

### Supplementary Note 2

The training device according to Supplementary note 1,
wherein the training means outputs a parameter group for each of the plurality of sub-models, and
wherein the integration means integrates the parameter groups of the plurality of sub-models by calculation.

### Supplementary Note 3

The training device according to Supplementary note 2, wherein the calculation is any of averaging, simple addition, weighted addition, and majority decision.

### Supplementary Note 4

The training device according to Supplementary note 1, wherein the models having the common architecture are models that are identical in a number of neurons, a number of layers, an array of parameters in layers, a combination of the layers, and a hyperparameter.

### Supplementary Note 5

The training device according to Supplementary note 1,
wherein the loss function includes a distribution-considered loss function considering a probability distribution of training data, and
wherein the training means calculates, for a model using the distribution-considered loss function, a difference between an estimated value by the model and a true value of training data, calculates a probability density function from the true value to obtain the probability distribution, and calculates a loss by weighting the difference with a weight based on the probability distribution.

### Supplementary Note 6

The training device according to Supplementary note 5, wherein the training means performs, for the model using the distribution-considered loss function, weighting with a larger weight for the difference related to the true value at a position farther from a center in the probability distribution.

### Supplementary Note 7

The training device according to Supplementary note 5, wherein the training means calculates, for the model using the distribution-considered loss function, a power in a Minkowski distance based on a parameter of the probability density function, and calculates the loss based on the difference and the power.

### Supplementary Note 8

The training device according to Supplementary note 1, wherein the sub-models and the final model are deep learning models that predict a disease risk from data regarding health of a person.

### Supplementary Note 9

A training method executed by a computer, comprising:
training models having a common architecture by using different loss functions to generate a plurality of sub-models; and
integrating the plurality of sub-models to generate a final model.

### Supplementary Note 10

A program for causing a computer to execute processing including:
training models having a common architecture by using different loss functions to generate a plurality of sub-models; and
integrating the plurality of sub-models to generate a final model.

Some or all of the configurations described in Supplementary Notes 2 to 8 dependent on the above-described Supplementary Note 1 can also be dependent on Supplementary Notes 9 and 10 by a dependency relationship similar to that of Supplementary Notes 2 to 8. Some or all of the configurations described as the Supplementary Notes can be similarly dependent on not only the Supplementary Notes 1, 9, and 10, but also diverse pieces of hardware and software, various recording means for recording software, or systems without departing from the above-described example embodiments.

While the present disclosure has been described with reference to the example embodiments and examples, the present disclosure is not limited to the above example embodiments and examples. Various changes which can be understood by those skilled in the art within the scope of the present disclosure can be made in the configuration and details of the present disclosure.

### DESCRIPTION OF SYMBOLS

5 Integration unit
11 Processor
20, 20x Training unit
21 Estimation unit
22 Difference calculation unit
23 Distribution calculation unit
24 Power calculation unit
25 Loss calculation unit
26 Optimization unit
100, 100x Training device

1. A training device comprising:
a memory configured to store instructions; and
one or more processors configured to execute the instructions to:
train models having a common architecture by using different loss functions to generate a plurality of sub-models; and
integrate the plurality of sub-models to generate a final model.

2. The training device according to claim 1,
wherein the one or more processors output a parameter group for each of the plurality of sub-models, and
wherein the one or more processors integrate the parameter groups of the plurality of sub-models by calculation.

3. The training device according to claim 2, wherein the calculation is any of averaging, simple addition, weighted addition, and majority decision.

4. The training device according to claim 1, wherein the models having the common architecture are models that are identical in a number of neurons, a number of layers, an array of parameters in layers, a combination of the layers, and a hyperparameter.

5. The training device according to claim 1,
wherein the loss function includes a distribution-considered loss function considering a probability distribution of training data, and
wherein the one or more processors calculate, for a model using the distribution-considered loss function, a difference between an estimated value by the model and a true value of training data, calculate a probability density function from the true value to obtain the probability distribution, and calculate a loss by weighting the difference with a weight based on the probability distribution.

6. The training device according to claim 5, wherein the one or more processors perform, for the model using the distribution-considered loss function, weighting with a larger weight for the difference related to the true value at a position farther from a center in the probability distribution.

7. The training device according to claim 5, wherein the one or more processors calculate, for the model using the distribution-considered loss function, a power in a Minkowski distance based on a parameter of the probability density function, and calculate the loss based on the difference and the power.

8. The training device according to claim 7, wherein the parameter of the probability density function is a distance of the true value from a center of the probability distribution.

9. The training device according to claim 8, wherein one or more processors calculate the power using a first hyperparameter that controls a maximum value of the power.

10. The training device according to claim 8, wherein one or more processors calculate the power using a second hyperparameter that controls rising rate of the power.

11. The training device according to claim 8, wherein one or more processors calculate the power using a third hyperparameter that indicates the power in a case where the distance of the true value from the center of the probability distribution is 0.

12. The training device according to claim 7, wherein the power corresponds the weight in the distribution-considered loss.

13. The training device according to claim 1, wherein the sub-models and the final model are deep learning models that predict a disease risk from data regarding health of a person.

14. A training method executed by a computer, comprising:
training models having a common architecture by using different loss functions to generate a plurality of sub-models; and
integrating the plurality of sub-models to generate a final model.

15. A non-transitory computer-readable recording medium storing a program, the program causing a computer to execute processing including:
training models having a common architecture by using different loss functions to generate a plurality of sub-models; and
integrating the plurality of sub-models to generate a final model.
